(19) United States
(12) Patent Application Publication
(10) Pub. No.: US 2015/0056618 A1
(43) Pub. Date: Feb. 26, 2015

(54) METHOD AND APPARATUS

(71) Applicant: BASE4 INNOVATION LTD, Cambridge, Cambridgeshire (GB)

(72) Inventors: Cameron Alexander Frayling, Cambridge (GB); Boris Breiner, Cambridge (GB); Michele Amasio, Cambridge (GB); Alessandra Natale, Cambridge (GB)

(73) Assignee: BASE 4 INNOVATION LTD., Cambridge, Cambridgeshire (GB)

(21) Appl. No.: 14/385,179

(22) PCT Filed: Mar. 15, 2013
(86) PCT No.: PCT/GB2013/050658

§ 371 Date: Sep. 15, 2014

(30) Foreign Application Priority Data
Mar. 16, 2012 (GB) 1204726.2

(51) Int. Cl. C12Q 1/68

(52) U.S. Cl. CPC C12Q 1/683
USPC 435/6.11; 435/287.2

(57) ABSTRACT

A method for mapping the number and location of restriction enzymes sites for a given restriction enzyme in a target nucleic acid comprises the steps of (1) translocating a target nucleic acid having detectable elements characteristic of the presence of the restriction enzyme sites therein through an analysing device comprising a nanopore and a detection window and (2) causing the detectable elements to be detected as they pass though the detection window. Typically the detectable elements are formed by attaching to the restriction enzyme sites a restriction enzyme to which one or more marker moieties have been added. The data or signal obtained from the detection is suitably in the form of a distribution profile of the detectable elements, and therefore the restriction enzyme sites along the length of the target nucleic acid and can be used to create a reference set of like distribution profiles against which new distributions can be compared. When the target nucleic acid is for example double stranded human DNA such comparisons enable valuable insights to be drawn about an individual's identity or his or her susceptibility to certain health conditions.

### METHOD AND APPARATUS

The present invention relates to a method and apparatus for characterising a nucleic acid in terms of the number and location of the restriction enzyme sites for a particular restriction enzyme present therein.

Restriction enzymes are enzymes, typically of bacterial or archeal origins, widely used in applications such as chromosomal mapping, DNA fingerprinting and genetic linkage mapping. They work by recognising certain defined oligonucleotide sequences (typically 4 to 12 nucleotides long) and their inverse in nucleic acid samples, e.g. double stranded DNA, and selectively making a cut in the sugar-phosphate backbone of each strand at occurrence thereof (the restriction enzyme site) to create a plurality of double stranded fragments characteristic of the original. Typically, once created these fragments are separated and identified using techniques such as gel electrophoresis.

WO2011074960 discloses a method for de novo whole genome sequencing based on a (sequence-based) physical map of a DNA sample clone bank based on end-sequencing tagged adapter-ligated restriction fragments, in combination with sequencing adapter-ligated restriction fragments of the DNA sample wherein the recognition sequence of the restriction enzyme used in the generation of the physical map is identical to at least part of the recognition sequence of the restriction enzyme used in the generation of the DNA sample.

U.S. 2008/0242556 discloses a method for characterizing one or more macromolecules using a nanofluidic device which involves translocating at least a portion of at least one region of the macromolecule through a fluidic nanochannel segment disposed substantially parallel to the surface of a substrate, wherein the fluidic nanochannel segment is capable of containing and elongating at least a portion of a region of the macromolecule, and has a cross-sectional dimension of less than about 1000 nm and a length of at least about 10 nm; monitoring, through a viewing window capable of permitting optical inspection of at least a portion of the contents of the fluidic nanochannel segment, one or more signals related to the translocation of one or more regions of the macromolecule through the nanochannel; and correlating the monitored signals to one or more characteristics of the macromolecule. In this method, the macromolecule only passes through the nanopore and not the detection window; the macromolecule is simply viewed through the detection window.

Dorvel et al (Nucleic Acids Research 37(12): 4170-4179, 2009) relates to analyzing the forces binding a restriction endonuclease to DNA using a synthetic nanopore. The authors used a synthetic nanopore to analyze how EcoPJ binds to its DNA target sequence in the absence of a Mg²⁺ ion cofactor. Using this method, they determined the strength of binding of each base in the target sequence to the protein.

An earlier paper by the same authors, Zhao et al (Nano Letters 7(6), 1680-1685, 2007) relates to detecting SNPs using a synthetic nanopore. The authors discovered a voltage threshold for permeation of dsDNA bound to a restriction enzyme through a synthetic nanopore. They found that a single mutation in the recognition site, i.e. a SNP, can be detected as a change in this threshold voltage and so it is possible to discriminate between SNPs by measuring threshold voltage in a synthetic nanopore.

U.S. 2010/0044211 discloses an apparatus for the detection of one or more target molecules which comprises a membrane that separates a first chamber and a second chamber, wherein the membrane comprises a nanochannel that is configured to allow passage of the target molecule(s), an electrical detection unit configured to detect the passage of the target molecule(s) through the nanochannel and an optical detection unit configured to identify the one or more target molecules passing through the nanochannel. Also disclosed is a method of detecting one or more target molecules which comprises applying an electrical source across such a membrane, detecting an electrical signal change upon passage of the target molecule(s) through the nanochannel, applying an electromagnetic energy source to the target molecule(s) and detecting an optical signal from the target molecule(s) generated by the electromagnetic energy source.

In order to cause cleavage of the DNA substrate many restriction enzymes require the presence of magnesium (II) cations for their activation so that in the absence thereof they simply reversibly bind to the restriction enzyme site (see for example Katsura et al. J. Biosci. Bioeng. 98(4), 293-7 (2004)). This has suggested to us the possibility of using them as markers for the presence of particular oligonucleotide sequences in long double stranded nucleic acid samples such as mammalian, especially human DNA; especially if the restriction enzyme itself is labelled with a moiety which can be analysed directly by physical means without requiring the DNA sample to be chemically digested into fragments the information content of which then needs to be reassembled.

According to the present invention, there is therefore provided a method for mapping the number and location of restriction enzyme sites for a given restriction enzyme in a target nucleic acid which comprises the steps of (1) translocating a target nucleic acid having detectable elements characteristic of the presence of the restriction enzyme site therein through an analysing device comprising a nanopore and a detection window and (2) causing the detectable elements to be detected as they pass though the detection window. In this method, the detectable elements are suitably detected so as to output data or a signal in the form of a distribution profile of the detectable elements along the length of the target nucleic acid. The distribution profiles so obtained can be used as is or added to a database of like profiles so that over time an extensive reference set is built up which constitutes a valuable research tool enabling genetic, biochemical and therapeutic conclusions and insights to be drawn therefrom.

The term “nucleic acid” as used herein means a polymer of nucleotides. Nucleotides themselves are also sometimes referred to as bases (in single stranded nucleic acid molecules) or as base pairs (in double stranded nucleic acid molecules) in an interchangeable fashion. Nucleic acids suitable for use in the method of the present invention are typically the naturally-occurring nucleic acids DNA, RNA or synthetic versions thereof. However the method can also be applied if desired to analogues such as PNA (peptide nucleic acid), LNA (locked nucleic acid), UNA (unlocked nucleic acid), GNA (glycol nucleic acid) and TNA (threose nucleic acid). The nucleic acids themselves in turn suitably comprise a sequence of at least some of the following nucleotides: adenine (A), cytosine (C), guanine (G), thymine (T) and uracil (U) 4-acetylcytidine, 5-(carboxyhydroxylmethyl)uridine, 2-O-methylcytidine, 5-carboxymethylaminomethyl-2-thiouridine, 5-carboxymethyl amino-methyluridine, dihydrouridine, 2-O-methylpseudouridine, 2-O-methylguanosine, inosine, N6-isopentyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, 5 -methoxyuridine, 5-methoxycarbonylmethyl-2-thiouridine, 5-methoxycarbonylmethyluridine, 2-methylthio-N6-isopentenyladenosine, uridine-5-oxyacetic acid-methylester, uridine-5-oxyacetic acid, wybutoxosine, wybutosine, pseudouridine, queuosine, 2-thiocytidine, 5-methyl-2-thiouridine, 2-thiouridine, 4-thiouridine, 5-methyluridine, 2-O-methyl-5-methyluridine and 2-O-methyluridine. Especially suitable nucleic acids are naturally occurring double stranded DNAs preferably mammalian DNAs most preferably of all human DNA.

Typically, the length of the target nucleic acid sequence is expressed in terms of the number of nucleotides it contains. For example, the term “kilobase” (kb) means 1000 nucleotides whilst “megabase” (Mb) means 1,000,000 nucleotides. The target nucleic acid used in the method of the present invention can in principle contain any number of nucleotides up to and exceeding the number typically found in a human or other mammalian gene. However the method of the present invention is also applicable to smaller polynucleotide fragments (e.g. fragments of a human gene) which are at least 10 bases (for single stranded nucleic acids) or base pairs (for double stranded nucleic acids) long, more typically at least 20, 30, 40, 50, 60, 70, 80, 90, 100, 500 or more bases/base pairs long or 1 kb, 2 kb, 5 kb, 10 kb, 20 kb, 50 kb, 100 kb, 250 kb, 500 kb or up to 1 Mb or more long. The target nucleic acid itself may be derived directly or indirectly from any available biological sample including but not limited to materials such as blood, sputum or urine.

It is a feature of the method of the present invention that the target nucleic acid is further comprised of detectable elements characteristic of the presence of restriction enzyme site for a given restriction enzyme. Suitably these detectable elements are derived from the reversible binding of a particular restriction enzyme, preferably modified with a marker moiety, to its corresponding restriction enzyme site. Typically, the detectable elements will all be of the same type for a given nucleic acid sample, although multiple types of detectable elements can be used and/or a given target nucleic acid may be analysed multiple times using different detectable elements if so desired. Furthermore the restriction enzyme can have more than one marker moiety attached thereto. In fact it may be beneficial in certain circumstances to map the same target nucleic acid in more than one of these ways to ensure a large number of different restriction enzyme sites are identified. Suitably, the detectable elements are such that they are able to generate, either directly or indirectly, corresponding characteristic data stream and/or a signal when caused to pass through the detection window. In one preferred embodiment of the invention, this characteristic data stream and/or signal is generated by the emission of photons characteristic of the detectable element fluorescing and/or Raman scattering incident light within the detection window. In the case of Raman scattering the detectable element will typically form part of the molecular structure of the modified restriction enzyme employed.

Suitably, the detectable elements are generated by attaching a restriction enzyme modified with a marker moiety to the restriction enzyme site. This attaching is carried out under conditions such that the nucleic acid backbone is not cleaved i.e. in the absence of entities which activate and/or catalyse this cleavage reaction. Preferably this is achieved by using a restriction enzyme which requires the presence of magnesium (II) cations to facilitate cleavage and carrying out the attachment in the absence of effective amounts of such cations. Restriction enzymes which exhibit this characteristic are well known in the art and for example include members of the so called Type I, Type II, and Type III families for example EcoRI, EcoRII, BamHI, HindIII as well as other suitable examples such as EcoRV.

The marker moiety can in principle be attached to the restriction enzyme by either physical or chemical means and, in the case of the latter, by covalent, ionic, dative bonding or ligation. One suitable class of marker moieties are those that are able to fluoresce such as xanthenes e.g. fluorescein, rhodamine and derivatives such as fluorescein isothiocyanate, rhodamine B and the like; coumarin derivatives, e.g. hydroxy-, methyl- and aminocoumarin, and cyanines such as Cy2, Cy3, Cy5 and Cy7. Preferred examples of this class are those marker moieties derived from the following commonly used dyes: Alexa dyes, cyanine dyes, Atto Tec dyes, and rhodamine dyes. Examples include: Atto 633 (ATTO-TEC GmbH), Atto 740 (ATTO-TEC GmbH), Rose Bengal, Alexa Fluor™ 750 C₅-maleimide (Invitrogen), Alexa Fluor™ 532 C₂-maleimide (Invitrogen), Cy3B maleimide and Rhodamine Red C₂-maleimide and Rhodamine Green. A second class of marker moieties are those able to Raman scatter incident light at a characteristic frequency which is capable of detection amongst any other Raman scattering events produced during the detection. Both classes of marker moieties can each be attached to the restriction enzyme using chemical techniques known in the art.

If the distribution profile characteristic of the target nucleic acid is to be compared against a reference set of pre-determined profiles characteristic of known nucleic acid samples, this can be done using either best fit statistical methods or visual inspection. Typically, however the comparison is performed computationally and can be based on a set of logic decision rules, or on a range of regression and classification methods (linear or not), or on pattern matching and machine learning methods (such as neural networks, kernel methods or graphical models). For example, the comparison can be performed by a computer that has a database or reference set of distribution profiles for known nucleic acids and a memory containing instructions which, when executed by the processor, compare the distribution profile of the target nucleic acid to the reference set. In the case where no matching is found the target nucleic acid can be added to the reference set for future reference if so desired.

In the method of the present invention, the target nucleic acid having the necessary detectable elements is analysed by translocating it through an analysing device comprising a nanopore having a detection window. In the method, the target nucleic acid is translocated through both the nanopore and the detection window. Preferably this detection window is defined by a localised electromagnetic field generated by plasmon resonance. In such an embodiment, the interaction between this electromagnetic field, the detectable elements and incident electromagnetic radiation impinging on the detection window is used to generate an increased level of fluorescence or Raman scattering which can be easily detected and analysed. One example of such an analysing device can be found in our WO 2009/030953 the contents of which are incorporated herein by reference. Briefly, this analysing device comprises a nano-perforated substrate separating sample providing and receiving chambers. The nano-perforated substrate may either be fabricated from an inorganic insulator or from organic or biological material. Preferably the nano-perforated substrate is an inorganic insulator such as a silicon carbide wafer. Typically, the nanopore is between 1 nm and 100 nm in diameter preferably 1 nm to 30 nm, 1 nm to 10 nm, 1 nm to 5 nm or 2 nm to 4 nm. The target nucleic acid is suitably caused to translocate from the sample to the receiving chambers via the nanopore by electrophoresis. Passage through the nanopore ensures that the target nucleic acid translocates in a coherent, linear fashion so that it emerges from the outlet thereof in a nucleotide by nucleotide fashion enabling the detectable elements and therefore the restriction enzyme sites to be detected in sequence.

The analysing device is suitably provided with a detection window juxtaposed either within the nanopore or adjacent its outlet. Typically this detection window is defined by one or more metallic moieties fabricated from gold or silver capable of undergoing plasmon resonance under the influence of incident electromagnetic radiation from a coherent source such as a laser. This plasmonic resonance generates the strong localised electromagnetic field through which the target nucleic acid passes. The exact geometry of these metallic moieties determines the geometry of the detection window and hence affects the nature of the interaction with the detectable elements. For example, the geometry of the detection window can be chosen so as to be optimised for increased photon emission, rather than for lateral localisation. This is achieved by producing detection windows with a greater z length (the dimension along which the nucleic acid translocates), and modifying their geometry appropriately in the x and y dimensions in order to ensure their peak plasmonic resonance frequency is maintained at a desired wavelength. Preferably, the detection window is sized so that the length in the z dimension is from 1 to 100 preferably from 10 to 50 nanometres.

The signal generated by the interaction of the detectable elements and the electromagnetic field can be detected by a detector such as a photocounter in the case of fluorescence or a spectrometer in the case of Raman scattering. The output of such a device will typically be an electrical signal characteristic of the target nucleic acid's distribution profile of the restriction enzyme sites of a given restriction enzyme.

The method of the present invention may suitably employ multiple detectors and multiple analysing devices. For example, an array of pairs of detectors and analysing devices may be used with each detector being arranged to detect photons generated using its paired analysing device. Other detectors including other detectors for detecting fluorescence such as a photomultiplier or single photon avalanche diode may be used.

In another preferred analysing method, the characteristic data stream and/or signal is generated by fluctuations in an electrical property of the detection window and/or its contents (e.g. changes in voltage, resistance or current flow occasioned by the detectable element blocking or enabling the flow of ions in the nucleic acid's associated translocation medium between electrodes). In this latter case, it may be possible through careful choice of the restriction enzyme to avoid having to label the same with a marker moiety. This embodiment of the invention is therefore typically carried out as an alternative to optical detection using, for example, fluorescence or Raman scattering, and not in addition to optical detection. Preferred translocation media used here are aqueous alkali metal electrolytes such as an aqueous potassium or sodium halide, nitrate or sulphate solution.

In a further aspect of the present invention there is provided an apparatus for identifying a target nucleic acid comprising detectable elements characteristic of the restriction enzyme sites of a given restriction enzyme the apparatus comprising: an analysing device comprising a nanopore having a detection window, wherein the analysing device is capable of plasmon resonance to produce a localised electromagnetic field which defines the detection window; a detector for detecting detectable elements of the target nucleic acid as they pass through the detection window to produce a distribution profile of the detectable elements along the target nucleic acid; and optionally a computer system for comparing the distribution profile to a reference set of distribution profiles for known nucleic acids. The computer system typically comprises a memory and a processor. Computer executable instructions can be provided which when executed by the processor compare the distribution profile of the target nucleic acids to a reference set of distribution profiles to identify the target nucleic acid or other relationships between it and the data in the database.

The present invention will now be exemplified by the following figures in which:

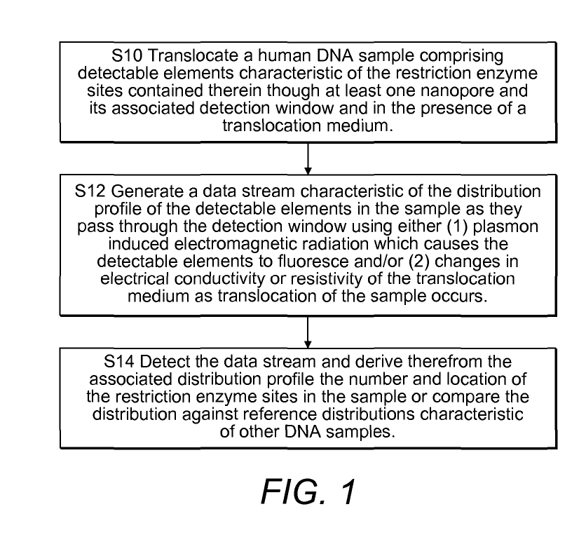

FIG. 1 is a flow diagram showing a method in accordance with an aspect of the present disclosure;

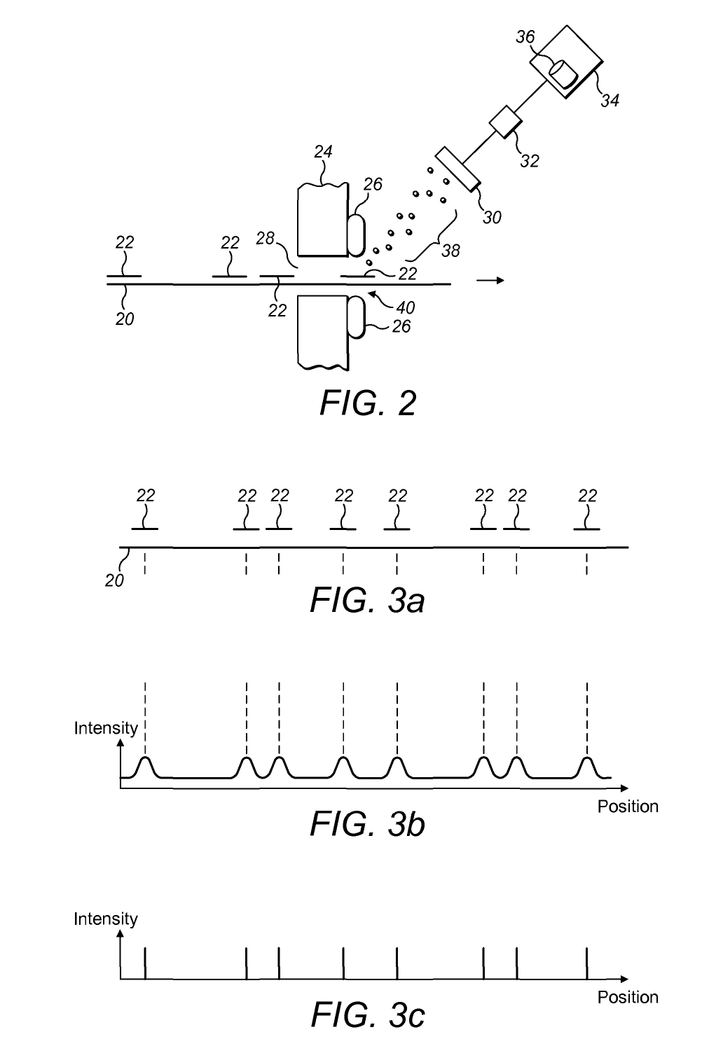

FIG. 2 schematically illustrates an apparatus for the method of FIG. 1 and

FIG. 3 illustrates the evolution of a schematic distribution profile for the target DNA analysed in the apparatus of FIG. 2.

FIG. 1 represents a flow diagram showing a method in accordance with the present invention. In one example, the method comprises, at step S10, translocating a target nucleic acid of human origin having detectable elements through a nanopore having a detection window. The nanopore is part of an analysing device which has a gold plasmonic structure that is capable of plasmon resonance under incident laser light to produce a localised electromagnetic field which defines the detection window. At step S12, the detectable elements are caused to fluoresce and are detected as they pass through the detection window to produce a distribution profile characteristic of the number and location of the restriction enzyme sites in the target nucleic acid. At step S14, the distribution profile of the target nucleic acid is compared against a reference set of distribution profiles.

FIG. 2 schematically illustrates an apparatus for performing the method of FIG. 1 comprising an analysing device 24, a photodetector 30, a data acquisition card 32 and a computer 34. 24 comprises a non-electrically conducting silicon carbide wafer perforated with a plurality of 4 nm diameter nanopores 28 and associated gold plasmonic structures 26 (doughnut shaped) juxtaposed over the outlet of 28 to define detection windows 40. In use, a human patient's DNA 20 (isolated from a blood sample) is for example first treated with a labelled EcoRV in the absence of magnesium (II) cations to generate detectable elements 22 and then caused to translocate though 28 and 40 by electrophoresis. In this example, the EcoRV has previously been labelled with Cy3B maleimide at position 58 on the enzyme (see for example Nucleic Acid Research, 36(12) 4118-4127 (2008)). 26 generate a localised electromagnetic field around the outlets of 28 which interacts with each 22 in turn causing them to fluoresce and emit photons 38 which are captured by 30. A laser (not shown) of frequency 750 nm and power 12 uW is used to induce the plasmon resonance in 26.

In an alternative detection method, 26 comprise one or more pairs of electrodes connected to each other via a battery and an ammeter (not shown) and the detectable elements created at the restriction enzyme sites are sized so as to interfere with the flow of ions between these electrodes arising from the sample's associated translocation medium (in this case aqueous potassium chloride). Specifically, in this embodiment, a potential difference is continuously applied across the electrodes and the resulting fluctuations in the current flowing between the electrodes (or any equivalent voltage fluctuations or changes in electrical resistance) are continuously monitored as a function of time and/or the progress of the translocation event to generate a data stream analogous to that described in the previous paragraph.

32 is used to receive the output of 30 and transfer it to 34 for analysis. The output is an electrical signal representing the distribution profile of the EcoRV restriction enzyme sites in the DNA in the form of a profile of fluorescence over the length of at least a portion of its length 20. 34 comprises a processor and memory connected to a central bus structure which is in turn connected to a display via a display adapter and one or more input devices (such as a mouse and/or keyboard). 34 further comprises a communications adapter which is also connected to the central bus. The communications adapter can receive communications, in particular communications containing new distribution profiles for new nucleic acid samples, which can be sent to the computer over a suitable communications link such as the internet.

The computer 34 has a database or reference set 36 of distribution profiles of restriction enzyme sites for known human DNA samples and the memory of the computer contains instructions which when executed by the processor compare the measured distribution profile of the target DNA sample (shown in FIG. 3) to this reference set to characterise the target DNA sample using pattern matching software.

1. A method for mapping the number and location of restriction enzyme sites for a given restriction enzyme in a target nucleic acid, the method comprising:
translocating a target nucleic acid having detectable elements characteristic of the presence of the restriction enzyme sites therein through an analysing device including a nanopore and a detection window; and
causing the detectable elements to be detected as the detectable elements pass though the detection window.

2. The method according to claim 1, further comprising
retrieving data and/or a signal characteristic of the distribution profile of the detectable elements, and therefore the restriction enzyme sites, in the target nucleic acid.

3. The method according to claim 2, further comprising
comparing the distribution profile with a reference set of known distribution profiles.

4. The method according to claim 1, wherein the analysing device is capable of plasmon resonance to produce a localised electromagnetic field which defines the detection window.

5. The method according to claim 1, wherein the detectable elements are formed by attaching a restriction enzyme including a marker moiety to the restriction enzyme site on the nucleic acid.

6. The method according to claim 2, wherein the distribution profile is generated by measuring fluctuations in an electrical property of the detection window and/or its contents.

7. The method according to claim 1, wherein the restriction enzyme is selected from the group consisting of Type I, Type II and Type III restriction enzymes and EcoRV.

8. The method according to claim 1, wherein the marker moiety is a fluorophore selected from the group consisting of xanthenes, coumarin derivatives and cyanine dyes.

9. The method according to claim 17, wherein the distribution profile is a profile of fluorescence over the length of at least a portion of the target nucleic acid, and wherein the plasmon resonance enhances fluorescent properties of the detectable elements.

10. The method according to claim 17, wherein the marker moiety can cause Raman scattering of light in a way characteristic of the marker moiety.

11. The method according to claim 10, wherein the distribution profile is a profile of Raman scattering over the length of at least a portion of the target nucleic acid,. and wherein the plasmon resonance increases the level of Raman scattering from the detectable elements.

12. The method according to claim 1, wherein the nanopore is located in a nano-perforated substrate which is an inorganic insulator.

13. The method according to claim 1, wherein the detection window is between 1 nanometre and 100 nanometres

14. The method according to claim 1, wherein the detection window is between 10 and 50 nanometres.

15. An apparatus for mapping the number and location of restriction enzyme sites for a given restriction enzyme in a target nucleic acid, the apparatus comprising:
an analysing device including a nanopore having a detection window, wherein the analysing device is capable of plasmon resonance to produce a localised electromagnetic field which defines the detection window; and
a detector for detecting detectable elements characteristic of the restriction enzyme sites in the target nucleic acid as the detectable elements pass through the detection window to produce a distribution profile of the detectable elements along the length of the target nucleic acid.

16. The apparatus according to claim 15, further comprising
a computer system for comparing the distribution profile to a reference set of distribution profiles for nucleic acids, or a means for attaching the computer system thereto.

17. The method according to claim 2, wherein the analysing device is capable of plasmon resonance to produce a localised electromagnetic field which defines the detection window.

18. The method according to claim 2, wherein the detectable elements are formed by attaching a restriction enzyme including a marker moiety to the restriction enzyme site on the nucleic acid.

19. The method according to claim 18, wherein the distribution profile is a profile of fluorescence over the length of at least a portion of the target nucleic acid, and wherein the plasmon resonance enhances fluorescent properties of the detectable elements.

20. The method according to claim 18, wherein the marker moiety can cause Raman scattering of light in a way characteristic of the marker moiety.
